(19) United States
(12) Patent Application Publication
(10) Pub. No.: US 2004/0152070 A1
(43) Pub. Date: Aug. 5, 2004

(54) Method of detection of HCV antibodies in combination assay or sole antibody assay

(76) Inventors: Dinesh O. Shah, Libertyville, IL (US); Yu Cheng, Mundelein, IL (US); James L. Stewart, Libertyville, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/357,816

(22) Filed: Feb. 4, 2003

(51) Int. Cl. C12Q 1/70; G01N 33/537; G01N 33/543; G01N 33/53

(52) U.S. Cl. 435/5; 435/7.1; 435/7.92

(57) ABSTRACT

The subject invention relates to methods for the simultaneous detection of Hepatitis C Virus (HCV) antigens as well as antibodies produced in response to HCV antigens. Such methods may be carried out in the presence of a diluent comprising a reductant or lacking a reductant. Furthermore, the performance of such methods may be maximized by altering such variables as the nature of the antigen coated on the solid phase, temperature application and time.

### BACKGROUND OF THE INVENTION

1. Technical Field

The subject invention relates to an improved method for the detection of Hepatitis C Virus (HCV) antibodies, whether said antibodies are being detected in a combination assay (which detects both HCV antigens and antibodies) or in an assay which detects only HCV antibodies.

2. Background Information

Recent epidemiological studies indicate that HCV infects more than 170 million people worldwide and that, in more than 50% of the cases, the infection is chronic. In the United States, there are approximately 4 million people infected, and 30,000 new infections are estimated to occur annually (NIH Conference, Hepatology Suppl 1:2S (1997)). In addition, HCV is responsible for 8,000-10,000 deaths annually in the United States and is the leading indicator for liver transplantation.

The HCV genome is a single-stranded RNA molecule of positive polarity that is approximately 9400-9500 nucleotides in length. The organization of the coding regions resembles that of other flaviviruses [Major et al., Hepatology 25:1527 (1997)] as well as the more recently discovered GB viruses [Muerhoff AS, et al., J Virol 69:5621 (1995)]. The HCV genome possesses a large open reading frame (ORF) encoding a polyprotein precursor of 3010 to 3033 amino acids depending on the particular isolate [Choo et al., Proc Natl Acad Sci USA 88:2451 (1991); Grakoui et al., J Virol 67:1385 (1993)]. HCV structural genes (core and envelope) are encoded near the 5′-end of the genome, followed by the proteases and helicase, the helicase cofactor and the replicase. Noncoding regions (NCR), thought to be important in replication, are found at each end of the genome.

HCV infection occurs primarily through parenteral exposure, i.e., through shared needles, by tattooing, or through transfusion of contaminated blood or blood products. Following exposure, the virus enters a susceptible hepatocyte and viral replication occurs. There is an eclipse phase period of approximately 10 days during which time there is no evidence of viral presence (i.e., viral RNA cannot be detected), serum transaminase levels are within normal limits, and there is no evidence of an immune response to HCV [Busch et al., Transfusion 40:143 (2000)]. Typically, about 10 days following exposure, HCV RNA can be detected, often with viral loads between 100,000-120,000,000 HCV RNA copies per ml of serum. Several weeks later, there is typically an increase in ALT levels indicating inflammation of the liver; antibodies are detected an average of about 70 days after exposure.

One of the preventive measures employed to limit the spread of HCV infections is to screen blood for exposure to HCV, either by the detection of antibodies to HCV or by the detection of viral-specific molecules (e.g., HCV RNA or HCV core proteins) in serum/plasma. Blood or blood products derived from individuals identified as having been exposed to HCV, by these tests, are removed from the blood supply and are not utilized for distribution to recipients of blood products (see, e.g., U.S. Pat. No. 6,172,189). These tests may also be utilized in the clinical setting to diagnose liver disease attributable to HCV infection. Due to the unavailability of native, intact HCV virions, serologic antibody tests have relied on recombinant antigens or synthetic peptides, representing selected fragments of the viral polyprotein. The first generation anti-HCV screening tests were based on detection of antibodies directed against a recombinant protein (HCV genotype 1a) originating from sequences located in the nonstructural NS-4 protein (C100-3) [Choo et al., Science 244:359 (1989); Kuo et al., Science 244:362 (1989)]. The first generation assays failed to detect antibodies in approximately 10% of individuals having chronic HCV infection and up to 10-30% of individuals presenting with acute HCV infection. The second generation anti-HCV assays have incorporated recombinant proteins from three different regions of the HCV genome (HCV genotype 1a), including amino acid sequences from the core, NS3, and NS4 protein [Mimms et al., Lancet 336:1590 (1990); Bresters et al., Vox Sang 62:213 (1992)], allowing a marked improvement over the first generation tests in identifying HCV infected blood donors [Aach et al., N Engl J Med 325:1325 (1991); Kleinman et al., Transfusion 32:805 (1992)]. The second generation assays detect antibodies in close to 100% of chronic HCV cases [Hino K., Intervirology 37:77 (1994)] and in nearly 100% of the acute cases by 12 weeks post infection [Alter et al., N Engl J Med 327:1899 (1992); Bresters et al., Vox Sang 62:213 (1992)]. The third generation test includes a recombinant protein expressing amino acid sequences from the NS5 region, as well as antigens from the core, NS3 and NS4. Some studies have indicated a slight improvement in sensitivity in comparing the third generation tests to second generation tests [Lee et al., Transfusion 35:845 (1995); Courouce et al. Transfusion 34:790-795 (1994)], but this improvement is largely attributed to changes in the NS3 protein rather than the inclusion of NS5 [Courouce et al., Lancet 343:853 (1994)].

In general, the second and third generation HCV antibody tests detect exposure to HCV about 70 days after exposure. Since HCV establishes persistent, and in many cases lifelong infection, the detection of antibodies to HCV represents a very efficient method for determining exposure to HCV. However, antibody testing alone will frequently fail to detect HCV infected individuals during the first 70 days after exposure.

The existing HCV antigen tests rely on detecting the presence of the HCV core antigen in serum or plasma. The core (or nucleocapsid) protein comprises the first 191 amino acids of the polyprotein. Two different types of serologic assays have been developed which permit detection of HCV core antigens in serum. One assay format detects HCV core antigens in subjects prior to seroconversion and is utilized in screening blood donors, while the other assay format detects core antigens only in hepatitis C patients, regardless of their HCV antibody status and is utilized in clinical laboratories to diagnose exposure to HCV or to monitor antiviral therapy.

Recent data on samples obtained during the pre-seroconversion period indicate that the HCV antigen test detects exposure to HCV significantly earlier than antibody testing [Aoyagi et al., J Clin Microbiol 37:1802 (1999); Peterson et al., Vox Sang 78:80(2000); Dawson et al., Transfusion, SD161, 40(2000); Muerhoff et al., 7^{th} International Meeting on Hepatitis C virus and related viruses, Dec. 3-7, 2000], and represents an alternative to nucleic acid testing for detecting exposure to HCV during the pre-seroconversion period. The advantages of HCV antigen detection are that the test is rapid, simple, may not require sample extraction or other pretreatment, and is not as prone to handling errors (e.g., contamination) as may occur in the HCV RNA tests.

In clinical laboratories, the HCV antigen test has comparable sensitivity to the HCV DNA tests in detecting exposure to HCV in patients infected with different HCV genotypes [Dickson et al., Transplantation 68:1512 (1999)] and in monitoring antiviral therapy [Tanaka et al., Hepatology 32:388 (2000); Tanaka et al., J Hepatol 23:742 (1995)]. Thus, HCV core antigen tests present a practical alternative to HCV RNA for screening blood donors or for monitoring antiviral therapy.

The uniqueness of the current invention lies in its ability to detect HCV antibodies and HCV antigens simultaneously (see also International Application No. PCT/JP99/04129), in a reductant free environment. Thus, the combination test or “combo” assay utilizes antigen detection to identify exposure to HCV during the pre-seroconversion “window period” and antibody detection to identify exposure to HCV after seroconversion.

In particular, in accordance with the method of the present invention, anti-core antibody detection or sensitivity is far superior in the absence of reductant as opposed to in the presence of reductant (particularly in connection with p9 MB-18 and upon heat stress application during the detection method). However, the present invention also encompasses a method whereby a reductant is utilized in order to improve antibody detection. For example, anti-NS3 antibody detection is far superior in the presence of a reductant. Other variables may also be altered, either with a reductant or without, in order to improve antibody detection sensitivity.

All U.S. patents, patent applications (e.g, U.S. patent application Ser. No. 09/891,983 and U.S. patent application Ser. No. 10/173,480) and publications referred to herein are hereby incorporated in their entirety by reference.

### SUMMARY OF THE INVENTION

The subject invention encompasses a method of simultaneously detecting at least one Hepatitis C Virus (HCV) antigen and at least one HCV antibody (i.e., antibody to HCV antigen) in a test sample. In particular, this method comprises contacting the test sample with a) a mixture of at least one HCV antigen or fragment thereof coated on a solid phase, for a time and under conditions sufficient for the formation of antibody/antigen complexes, presence of the antibody/antigen complexes indicating presence of the at least one HCV antibody in the test sample; and

b) at least one HCV antibody or fragment thereof coated on the solid phase, for a time and under conditions sufficient for the formation of antigen/antibody complexes, presence of antigen/antibody complexes indicating presence of at least one HCV antigen in the test sample. The method is carried out in the absence of a reductant. The at least one HCV antigen coated on the solid phase (e.g., a microparticle) may be, for example, selected from core antigen, NS3, NS4, NS5, and fragments thereof. In particular, the at least one HCV antigen coated on the solid phase may comprises, for example, amino acids 1192-1457 of NS3 and amino acids 1-31 and 51-100 of core protein, and the at least one antibody detected by the antigen coated on the solid phase may be, for example, anti-NS3 antibody and/or anti-core antibody. Alternatively, the at least one HCV antigen coated on the solid phase may, for example, comprise amino acids 1192-1457 of NS3 and amino acids 1-31, 34-46 and 49-100 of core protein. When this antigen is utilized, the at least one antibody detected may be, for example, anti-NS3 antibody and/or anti-core antibody. (All amino acid sequences referred to herein are disclosed in U.S. patent application Ser. No. 10/173,480 herein incorporated in its entirety by reference.)

The above method may further comprise the step of applying heat (i.e., heat stress) subsequent to the contacting step. In this case, the at least one antigen coated on the solid phase may comprise, for example, amino acids 1192-1457 of NS3 and amino acids 1-31 and 51-100 of core protein, and the at least one antibody detected by the antigen coated on the solid phase may be, for example, anti-core antibody.

The present invention also encompasses another method for simultaneously detecting the presence of at least one HCV antigen and at least one HCV antibody in a test sample. This method comprises the steps of: a) contacting the test sample with: 1) at least one HCV antigen or fragment thereof coated on a solid phase, for a time and under conditions sufficient for the formation of antibody/antigen complexes and 2) at least one HCV antibody or portion thereof coated on the solid phase, for a time and under conditions sufficient for the formation of antigen/antibody complexes; b) adding a first conjugate to the resulting antibody/antigen complexes for a time and under conditions sufficient to allow the conjugate to bind to the bound antibody in (a)(i), wherein the conjugate comprises an antibody (e.g., mouse, anti-human IgG) attached to a chemiluminescent compound capable of generating a detectable signal and simultaneously adding a second conjugate to the resulting antigen/antibody complexes for a time and under conditions sufficient to allow the conjugate to bind to the bound antigen in (a)(2), wherein the conjugate comprises an antibody attached to the chemiluminescent compound capable of generating a detectable signal; and c) detecting the generated signal, presence of the signal indicating presence of at least one HCV antigen, at least one HCV antibody, or both, in the test sample. The method is carried out in the absence of a reductant. The antigen coated on the solid phase may be as described above, as well as the antibodies detected by this antigen. Further heat may also be utilized as noted above. Use of the antigen noted above may also be combined with the heat stress in order to detect the at least one antibody, noted above.

Additionally, the present invention includes a further method of simultaneously detecting at least one Hepatitis C Virus (HCV) antigen and at least one HCV antibody in a test sample. This method comprises contacting the test sample with a mixture of: 1) at least one HCV antigen or fragment thereof coated on a solid phase, for a time and under conditions sufficient for the formation of antibody/antigen complexes, presence of the antibody/antigen complexes indicating presence of the at least one HCV antibody in the test sample; and 2) at least one HCV antibody or fragment thereof coated on the solid phase, for a time and under conditions sufficient for the formation of antigen/antibody complexes, presence of antigen/antibody complexes indicating presence of at least one HCV antigen in the test sample, wherein the detection of the at least one HCV antigen and the at least one HCV antibody are carried out in the presence of a reductant selected from the group consisting of Dithiothreitol (DTT) and Cysteine. The at least one HCV antigen coated on the solid phase may be, for example, core antigen, NS3, NS4, NS5, or fragments thereof. More specifically, the at least one antigen coated on the solid phase may comprise, for example, amino acids 1192-1457 of NS3 and amino acids 1-31 and 51-100 of core protein, the least one antibody detected by the antigen may be, for example, anti-NS3 antibody or anti-core antibody, and the reductant may be, for example, DTT. Alternatively, the at least one antigen coated on the solid phase may comprise, for example, amino acids 1192-1457 of NS3 and amino acids 1-30, 34-46 and 49-100 of core protein. This antigen may be used to detect the at least one antibody, for example, anti-NS3 antibody and/or anti-core antibody, upon use of DTT as a reductant.

Heat may further be applied to the method described directly above, subsequent to the contacting step. When heat is applied, the at least one antigen coated on the solid phase comprises amino acids 1192-1457 of NS3 and amino acids 1-31 and 51-100 of core protein, the at least one antibody detected may be anti-NS3 antibody and/or anti-core antibody, and the reductant utilized is DTT.

Furthermore, the present invention also encompasses a method for simultaneously detecting the presence of at least one HCV antigen and at least one HCV antibody in a test sample comprising the steps of: a) contacting the test sample with: 1) at least one HCV antigen or fragment thereof coated on a solid phase, for a time and under conditions sufficient for the formation of antibody/antigen complexes and 2) at least one first HCV antibody or fragment thereof coated on the solid phase, for a time and under conditions sufficient for the formation of antigen/antibody complexes; b) adding a first conjugate to the resulting antibody/antigen complexes for a time and under conditions sufficient to allow the first conjugate to bind to the bound antibody in (a)(1), wherein the first conjugate comprises an antibody attached to a chemiluminescent compound capable of generating a detectable signal and simultaneously adding a second conjugate to the resulting antigen/antibody complexes for a time and under conditions sufficient to allow the second conjugate to bind to the bound antigen in (a)(2), wherein the conjugate comprises an antibody attached to said chemiluminescent compound capable of generating a detectable signal; and c) detecting the generated signal, presence of the signal indicating presence of the at least one HCV antigen, the at least one HCV antibody, or both, in the test sample, wherein detection of the at least one HCV antibody is carried out in the presence of a diluent comprising a reductant selected from the group consisting of DTT and Cysteine. Again, the at least one HCV antigen coated on the solid phase may be, for example, core antigen, NS3, NS4, NS5, or fragments thereof. In particular, the least one antigen coated on the solid phase may comprise, for example, amino acids 1192-1457 of NS3 and amino acids 1-31 and 51-100 of core protein, the at least one antibody detected by this antigen may be anti-NS3 antibody and/or anti-core antibody, and the reductant used may DTT. Alternatively, in the method, the at least one antigen coated on the solid phase may comprise, for example, amino acids 1192-1457 of NS3 and amino acids 1-30, 34-46 and 49-100 of core protein, the at least one antibody detected by this antigen may be, for example, anti-NS3 antibody and/or anti-core antibody, and the reductant utilized may be, for example, DTT.

A heat step may also be applied to this method after step (a). When this heat step is utilized, the at least one antigen coated on the solid phase may comprise, for example, amino acids 1192-1457 of NS3 and amino acids 1-31 and 51-100 of core protein, the at least one antibody detected by the antigen may be, for example, anti-NS3 antibody and/or anti-core antibody, and the reductant used may be, for example, DTT.

Additionally, the present invention encompasses a method of detecting at least one HCV antibody in a test sample comprising contacting the test sample with at least one HCV antigen or fragment thereof coated on a solid phase, for a time and under conditions sufficient for the formation of antibody/antigen complexes, presence of the antibody/antigen complexes indicating presence of the at least one HCV antibody in the test sample, wherein the detection of the at least one HCV antibody is carried out in the absence of reductant. The at least one antigen used to coat the solid phase may be as described above, and the antibody detected may be, for example, anti-NS3 antibody and/or anti-core antibody.

Heat may also be applied subsequent to the contacting step. When heat is utilized, the at least one antigen coated on the solid phase comprises, for example, amino acids 1192-1457 of NS3 and amino acids 1-31 and 51-100 of core protein and the at least one antibody detected is, for example, anti-core antibody.

Also, the present invention includes a method of detecting at least one HCV antibody in a test sample comprising the steps of: a) contacting the test sample with at least one HCV antigen or fragment thereof coated on a solid phase, for a time and under conditions sufficient for the formation of antibody/antigen complexes; b) adding a conjugate to the resulting antibody/antigen complexes for a time and under conditions sufficient to allow the conjugate to bind to the bound antibody in (a), wherein the conjugate comprises an antibody attached to a chemiluminescent compound capable of generating a detectable signal; and c) detecting the generated signal, presence of the signal indicating presence of the at least one HCV antibody in the test sample. Again, the method is carried out in the absence of a reductant. Once again, the at least one antigen coated on the solid phase may be as described above. In particular, the at least one antigen coated on the solid phase may comprise for example, amino acids 1192-1457 of NS3 and amino acids 1-31 and 51-100 of core protein and the at least one antibody detected may be, for example, anti-NS3 antibody and/or anti-core antibody. Alternatively, the at least one antigen coated on the solid phase may comprise, for example, amino acids 1192-1457 of NS3 and amino acids 1-30, 34-46 and 49-100 amino acids of core protein and the at least one antibody may be, for example, anti-NS3 antibody and/or anti-core antibody.

Again, this method may further comprise the step of applying heat subsequent to step (a). When heat is applied, the at least one antigen coated on the solid phase preferably comprises amino acids 1192-1457 of NS3 and amino acids 1-31 and 51-100 of core protein and the at least one antibody detected is anti-core antibody.

The present invention also encompasses a method of detecting at least one HCV antibody in a test sample comprising contacting the test sample with at least one HCV antigen or fragment thereof coated on a solid phase, for a time and under conditions sufficient for the formation of antibody/antigen complexes, presence of the antibody/antigen complexes indicating presence of the at least one HCV antibody in the test sample. The detection method is carried out in the presence of a reductant selected from the group consisting of DTT and Cysteine. In this method, the at least one HCV antigen coated on the solid phase may be, for example, core antigen, NS3, NS4 or NS5, and fragments thereof. In particular, the at least one antigen coated on the solid phase comprises, for example, amino acids 1192-1457 of NS3 and amino acids 1-31 and 51-100 of core protein. The at least one antibody detected may be, for example, anti-NS3 antibody and/or anti-core antibody, and the reductant may be, for example, DTT. Alternatively, the at least one antigen coated on the solid phase may comprise, for example, amino acids 1192-1457 of NS3 and amino acids 1-30, 34-46 and 49-100 of core protein. The at least one antibody detected may be, for example, anti-NS3 antibody and/or anti-core antibody, and the reductant is, for example, DTT.

In this method, one may apply heat stress subsequent to the contacting step. In this instance, the at least one antigen coated on the solid phase may comprise, for example, amino acids 1192-1457 of NS3 and amino acids 1-31 and 51-100 of core protein, the at least one antibody detected is, for example, anti-NS3 antibody and/or anti-core antibody, and the reductant is, for example, DTT.

Also, the present invention encompasses another method for detecting the presence of at least one HCV antibody in a test sample. This method comprises the steps of: a) contacting the test sample with at least one HCV antigen or fragment thereof coated on a solid phase, for a time and under conditions sufficient for the formation of antibody/antigen complexes; b) adding a conjugate to the resulting antibody/antigen complexes for a time and under conditions sufficient to allow the conjugate to bind to the bound antibody in (a), wherein conjugate comprises an antibody attached to a chemiluminescent compound capable of generating a detectable signal; and c) detecting the generated signal, presence of the signal indicating presence of at least one HCV antibody in the test sample. The method is carried out in the presence of a diluent comprising a reductant selected from the group consisting of DTT and Cysteine. Again, at least one HCV antigen coated on the solid phase may be, for example, core antigen, NS3, NS4, or NS5, or fragments thereof. In particular, the at least one antigen coated on the solid phase may comprise, for example, amino acids 1192-1457 of NS3 and amino acids 1-31 and 51-100 of core protein, the at least one antibody detected may be, for example, anti-NS3 antibody and/or anti-core antibody, and the reductant may be, for example, DTT. Alternatively, the at least one antigen coated on the solid phase may comprise, for example, amino acids 1192-1457 of NS3 and amino acids 1-30, 34-46 and 49-100 of core protein, the at least one antibody may be, for example, anti-NS3 antibody and/or anti-core antibody, and the reductant may be, for example, DTT.

This method may also further comprise the step of applying heat (i.e., heat stress) subsequent to step (a). When heat is applied, the at least one antigen coated on the solid phase may comprise, for example, amino acids 1192-1457 of NS3 and amino acids 1-31 and 51-100 of core protein, the at least one antibody may be anti-NS3 antibody and/or anti-core antibody, and the reductant is, for example, DTT.

The present invention also encompasses a kit comprising: a container containing at least one HCV antigen coated on a solid phase, wherein the container contains a diluent lacking a reductant; and a container containing at least one HCV antibody coated on a solid phase.

Additionally, the present invention includes a kit comprising a container containing: 1) at least one HCV antigen coated on a solid phase and 2) at least one HCV antibody, coated on the solid phase, wherein the container contains a diluent lacking a reductant.

### BRIEF DESCRIPTION OF THE DRAWINGS

FIG. 1 illustrates the Abbott PRISM® HCV Ab assay format. The assay uses a 2-step format that consists of microparticles coated with recombinant HCV antigens from the core, NS3, NS4 and NS5 regions of the HCV genome (see U.S. Pat. No. 5,705,330 for a discussion of microparticles coated with HC43, C100 or NS5). These microparticles, when combined with the donor specimen, a reductant-free diluent, and a complex of goat polyclonal anti-human F(ab′)2 fragment/murine monoclonal anti-biotin:Acridinium conjugate yield an amount of photons representing a qualitative measurement of anti-HCV antibodies in the specimen when triggered with the PRISM® Activator solution.

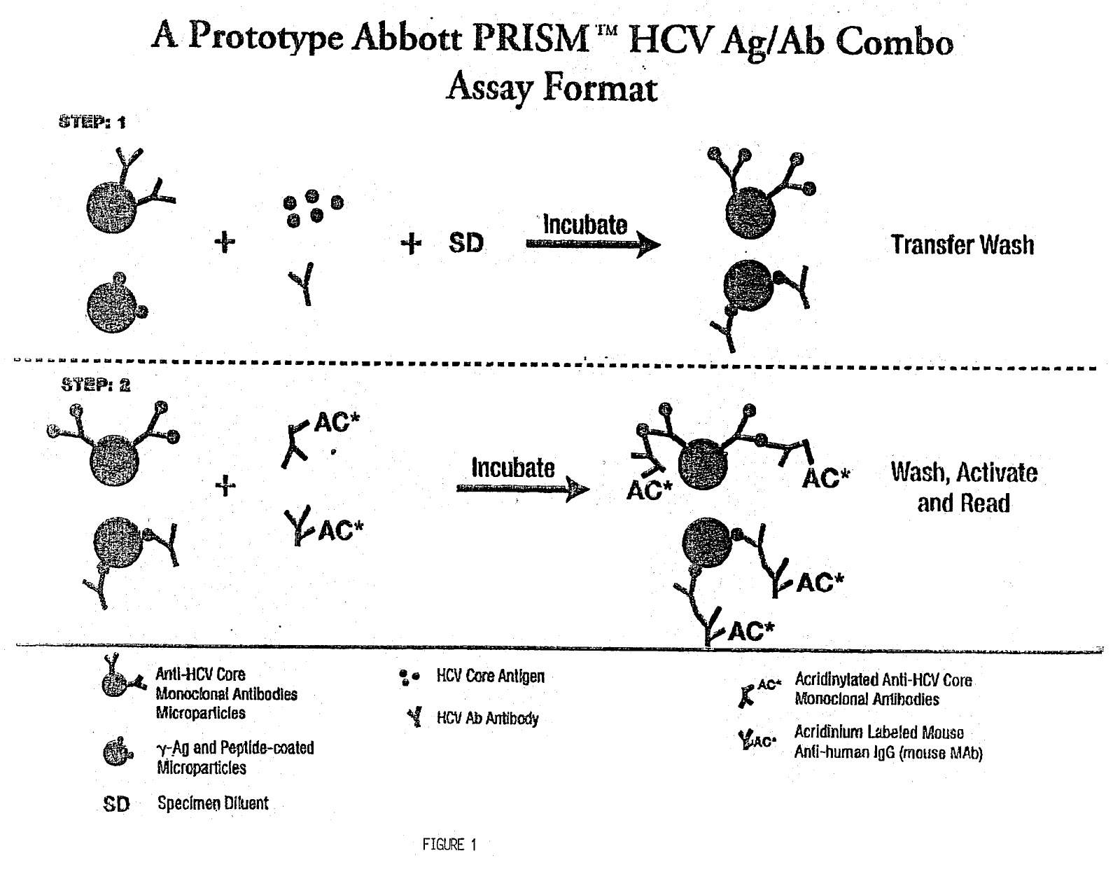

FIG. 2 illustrates the HCV Ag/Ab combo assay format. The assay uses a 2-step format. When HCV recombinant antigen and monoclonal antibody blended microparticles (e.g., HCV peptide from the core, and recombinant antigens from the NS3, NS4 and NS5 regions of the HCV genome blended with microparticles coated with c11-14) are combined with the donor specimen, a reductant-free diluent and blended Acridinium-labeled Mabs (e.g., acridinium labeled c11-10 and acridinylated mouse-anti-human IgG), an amount of photons representing a qualitative measurement of anti-HCV antigens or anti-HCV antibodies or both in the specimen will result when triggered with PRISM® Activator solution.

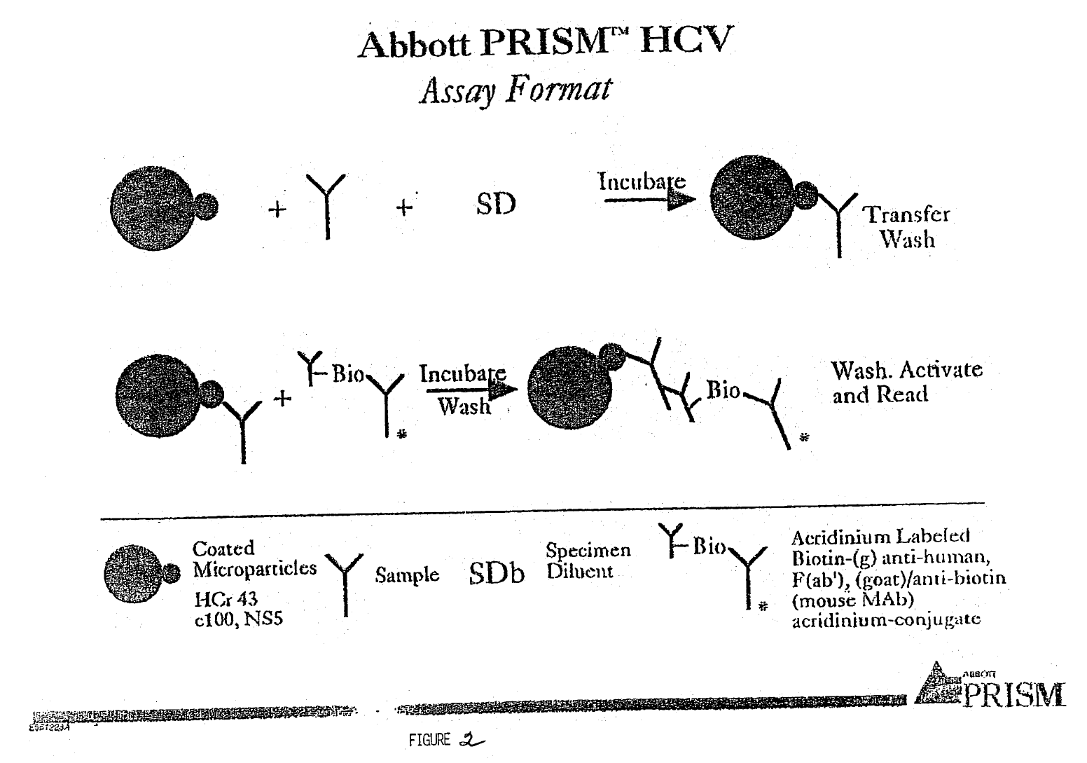

### DETAILED DESCRIPTION OF THE INVENTION

The subject invention relates to various methods which may be utilized in order to detect antigens of HCV and antibodies to HCV in a biological sample. These methods may be carried out in the presence of a reductant-free diluent or in the presence of a reductant-containing diluent. Thus, if an individual has either developed specific antibodies to HCV and/or has HCV specific antigens in the biological sample tested, the methods of the present invention will yield a positive result. Such results may be used, for example, to diagnose the patient in terms of presence and status of infection (i.e., acute or chronic) as well as to determine the suitability of a donor blood or blood product sample for transfusion.

Also, the present invention overcomes the problems associated with the “window period” (i.e., 50-60 days post infection) wherein an individual may be infected with HCV but may not have developed antibodies yet. Such individuals may transmit HCV to others during this period. Thus, by detecting HCV during this “window period”, the present invention allows for a quick diagnosis of HCV, as opposed to waiting for the development of antibodies, and prevents contamination of the blood supply.

In one embodiment of the present invention, HCV viral antigens (e.g., core, N3, N4 and N5), portions thereof, or mixtures of the full-length proteins or portions thereof are coated on a solid phase (or are in a liquid phase), in the presence of a reductant-free diluent or in the presence of a diluent comprising reeducating. The test or biological sample (e.g., serum, plasma, urine, etc.) is then contacted with the solid phase. If antibodies are present in the sample, such antibodies bind to the antigens on the solid phase and are then detected by either a direct or indirect method. The direct method comprises simply detecting presence of the complex itself and thus presence of the antibodies. In the indirect method, a conjugate is added to the bound antibody. The conjugate comprises a second antibody, which binds to the first bound antibody, attached to a signal-generating compound or label. Should the second antibody bind to a bound first antibody, the signal-generating compound generates a measurable signal. Such signal then indicates presence of the first antibody in the test sample.

Examples of solid phases used in diagnostic immunoassays are porous and non-porous materials, latex particles, magnetic particles, microparticles (see U.S. Pat. No. 5,705,330), beads, membranes, microtiter wells and plastic tubes. The choice of solid phase material and method of labeling the antigen or antibody present in the conjugate, if desired, are determined based upon desired assay format performance characteristics.

As noted above, the conjugate (or indicator reagent) will comprise an antibody (or perhaps anti-antibody, depending upon the assay), attached to a signal-generating compound or label. This signal-generating compound or “label” is itself detectable or may be reacted with one or more additional compounds to generate a detectable product. Examples of signal-generating compounds include chromogens, radioisotopes (e.g., 125I, 131I, 32P, 3H, 35S and 14C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Examples of biological fluids which may be tested by the above immunoassays include plasma, urine, whole blood, dried whole blood, serum, cerebrospinal fluid, saliva, tears, nasal washes or aqueous extracts of tissues and cells.

At the same time as the antibodies are being detected, HCV antigens are also being detected; thus, the present invention obviates the need for the running of two different tests. (Of course, an antibody assay, in the presence of a reductant-free diluent may be carried out alone if one is looking only for the presence of antibodies in a patient sample.) This is accomplished by exposing the test sample to a solid phase (or liquid phase) coated with specific antibodies to HCV (e.g., human or animal monoclonal antibodies to core, polyclonal antibodies, chimeric antibodies, etc.). Antigens, if present in the sample, bind to the solid phase and may then be detected by a direct or indirect method as described above. More specifically, the indirect method involves the addition of a conjugate comprising a second antibody (which binds to the bound antigen) attached to a label or signal-generating compound. When the second antibody binds to the bound antigen, a detectable signal is then generated indicating presence of HCV antigen in the test sample.

The antibodies which are coated on the solid phase as well as the “second antibody” may be, as noted above, monoclonal antibodies (e.g., mouse anti-human IgG) or polyclonal antibodies. For example, if one chooses to utilize monoclonal antibodies, they may be selected from Abbott monoclonal antibodies 13-959-270, 14-1269-281, 14-1287-252, 14-153-234, 14-153-462, 14-1705-225, 14-1708-269, 14-1708-403, 14-178-125, 14-188-104, 14-283-112, 14-635-225, 14-726-217, 14-886-216, 14-947-104 and 14-945-218. The following anti-core monoclonal antibodies may also be utilized for purposes of the present invention: 107-35-54, 110-81-17, 13-975-157, 14-1350-210 (see U.S. Pat. No. 5,753,430) and Tonen HCV core monoclonals C11-3, 7, 10, 14 and 15 (see PCT Application WO 099/06836), all of which are available from the American Type Culture Collection, 10801 University Boulevard, Manassas, Va. 20110-2209. (For a discussion of the manner in which monoclonal antibodies may be created, see Kohler and Milstein, Nature (1975) 256:494, and reviewed in Monoclonal Hybridoma Antibodies: Techniques and Applications, ed. Hurrell (CRC Press, Inc., 1982); see also J. W. Goding in Monoclonal Antibodies: Principles and Practice (Academic Press, N.Y., 1983; see also U.S. Pat. No. 5,753,430).

It should be noted that HCV core protein may be one possible target of the HCV antigen portion of the assay. More specifically, the detection of the core protein is accomplished by using monoclonal antibodies directed towards epitopes within the core protein. These anti-core monoclonals are placed on the solid phase and facilitate the capture of core antigen proteins from the test sample. For detection of HCV antibodies in the test sample, recombinant HCV core protein is also placed on the solid phase. It should be noted however that there are significant problems associated with the use of a single protein as the target for an antigen test and as the capture reagent for antibody detection, namely there is significant “cross-reactivity” between the core antigen and the anti-core monoclonal antibodies coated onto the solid phase(s). This results in a false positive signal, even in the absence of the test sample, since the monoclonal antibodies will bind to epitopes present on the recombinant protein.

In order to avoid such cross-reactivity, the core protein used in the antibody detection portion of the assay may be modified such that the ability of the anti-core monoclonals to bind HCV core is eliminated. Such modification may be achieved by use of recombinant DNA technology in which the epitope region (i.e., the short sequence of amino acids needed for monoclonal antibody binding) is eliminated or modified. Thus, use of the modified recombinant core protein would consequently maintain several human epitopes to which antibodies present in the serum of infected individuals would bind; however, the anti-core monoclonal antibodies used for antigen capture would not bind the modified protein. Alternatively, one could replace the HCV core recombinant protein with polypeptides that include sequences known to bind to antibodies present in the serum of most infected individuals, but do not include sequences containing the epitopes recognized by the anti-core monoclonals used to detect HCV core antigens.

More specifically, as noted above, in order to avoid cross-reactivity, one may use core antigens for antibody detection in the assay. In particular, in the present invention, the solid phase may be coated with nonstructural proteins (NS) 3, 4 and/or 5 (i.e., NS3, NS4 and/or NS5) and/or the core protein. (See U.S. Pat. No. 5,705,330 for the amino acid sequences of proteins discussed herein.) Alternatively, in the present invention, the solid phase may be coated with any of the above-mentioned full-length HCV proteins, or segments or portions thereof, either individually or in combination (for antibody detection). The antigens used for coating the solid phase may be generated as a contiguous recombinant protein, expressed as recombinant proteins, either as a single entity or as discrete entities, or as synthetic peptides designed either as a single entity or discrete entities.

It should also be noted that one may also detect antibodies to HCV E2 (or other antibodies to HCV antigens) in the combo assay. Thus, using the present assay described herein, one may replace an assay which detects anti-core antibody. Alternatively, one may supplement such an anti-core antibody assay with the antigen assay portion of the combo assay described herein. (See, e.g., U.S. Pat. No. 6,156,495 relating to detection of HGBV E2 antibody or antigen.)

With respect to detection of antigens in the present invention, as noted above, the monoclonal or polyclonal antibodies coated on the solid phase must not recognize the core antigens used on the solid phase (for antibody detection). Thus, for example, in the present invention, one may use the full antibody or a fragment thereof. (For purposes of the present invention, a “fragment” or “portion” of an antibody is defined as a subunit of the antibody which reacts in the same manner, functionally, as the full antibody with respect to binding properties.)

Additionally, it should also be noted that the initial capture antibody (for detecting HCV antigens) used in the immunoassay may be covalently or non-covalently (e.g., ionic, hydrophobic, etc.) attached to the solid phase. Linking agents for covalent attachment are known in the art and may be part of the solid phase or derivatized to it prior to coating.

The second manner in which to use the solid phase for detecting HCV antibodies involves elimination of the core antigens entirely. For example, the solid phase is coated with NS3, NS4 and/or NS5 and a substitute for the core protein or regions thereof (e.g., E2). In contrast, the antibodies coated on the solid phase for detection of antigen are directed against the core protein of HCV.

Other assay formats which may be used for purposes of the present invention, in order to simultaneously detect antigens and antibodies include, for example, Dual assay strip blots, a rapid test, a Western blot, as well as the use of paramagnetic particles in, for example, an Architect® assay (Frank Quinn, The Immunoassay Handbook, Second edition, edited by David Wild, pages 363-367, 2001). Such formats are known to those of ordinary skill in the art.

It should also be noted that the assays of the present invention may also be used to solely detect HCV antigens or HCV antibodies, rather than both, if desired, and the sole HCV antibody assay is preferably carried out in the presence of a diluent lacking a reductant. Certainly, if one desires to establish that an infection initially exists, one may simply want to determine the presence of antigen in a test sample such as during the “window period”. On the other hand, if one wants to establish the stage of infection (e.g., acute versus chronic), one may wish to look for the presence of antibodies and titer thereof.

It should also be noted that the elements of the assay described above are particularly suitable for use in the form of a kit. The kit may also comprise one container such as vial, bottles or strip, with each container with a pre-set solid phase, and other containers containing the respective conjugates. These kits may also contain vials or containers of other reagents needed for performing the assay, such as washing, processing and indicator reagents.

The present invention may be illustrated by the use of the following non-limiting examples:

### EXAMPLE I

Coating of Truncated HC43 Onto Microparticles

Polystyrene microparticles were equilibrated in microparticle coating buffer (i.e., 2-[N-Morpholino]ethanesulfonic acid (MES) buffer with Sodium dodecyl sulfate (SDS)) at pH 6.3, at 1% solid percentage for 15 minutes, and then truncated HC43 (p9 MB18 or p9 MB31)(see U.S. Pat. No. 5,705,350) was added at 50 ug/ml in a final concentration for passive coating (see U.S. patent application Ser. No. 10/173,480, enjoying common ownership with the owner of the present invention) for a discussion of the preparation of p9 MB18 and p9 MB31). The mixture was incubated at room temperature overnight with rotation.

The p9 MB18-coated or p9 MB31 coated microparticles were washed with microparticle washing buffer. The buffer contained phosphate, SDS, Ethylenediaminetetraacetic Acid (EDTA), Sodium Chloride, pH 6.5. After washing, microparticles were re-suspended with the same buffer and heat stressed at 56° C. overnight.

### EXAMPLE II

### Performance of HCV Antibody/Antigen Prototype Assay

The assay was performed by using a single channel PRISM® instrument (Abbott Laboratories, Abbott Park, Ill.) as described in the publication “Automated Panel Analyzers” by D. Shah and J. Stewart, (Immunoassay Handbook, 2^{nd} Edition, Ed. D. Wild, Nature Publishing, NY, N.Y.).

Briefly, 100 ul of sample; 50 ul of specimen diluent buffer (SDB) which was a borated saline with detergent, Bovine Albumin, superoxide dismutase (SOD), and 0.1% sodium azide; and 50 ul of microparticles coupled with p9 MB18 or p9 MB31 were added to an incubation well at Station 1. The reaction tray was moved to Station 4 with a lapse of 18 minutes. At Station 4, the reaction mixture was transferred to a reaction well by a transfer wash buffer that was a Tris saline with detergent and 0.1% Proclin 300. Microparticles in the reaction mixture were captured by fibrous matrix (which allows liquid to filter down) in the reaction well. At Station 5, 50 ul of conjugate containing an acridinium labeled mouse anti-human antibody was dispensed to the reaction well. The reaction tray was moved with a lapse of 22.3 minutes to Station 8, the reaction mixture in the reaction well was washed with final wash buffer which was a Borate buffer with detergent and 0.1% Proclin 300. The tray was then moved to Station 9 where the trigger solution, an alkaline hydrogen peroxide, was injected to the reaction well and its signal was measured by a photo-multiplier tube. The chemiluminescence counts from each reaction well were integrated and saved in alignment with its sample ID to the batch file.

Assay Controls: Negative Calibrator (NC) is a re-calcified normal human plasma which tested negative for HIV, HTLV, HBsAg, HBcore, and HCV.

### EXAMPLE III

### Testing Truncated HC43-Coated Microparticles Under Different Environments

The p9 MB18 and p9 MB31 coated microparticles were diluted to 0.025% in solid concentration with different microparticle diluents as follows: 1) without reductant, 2) with 2 mM Dithiothreitol (DTT), 3) with 3 mM Cysteine in the phosphate buffer containing Sucrose, Sodium Chloride, EDTA, SDS, pH 6.5. The diluted microparticles in the different buffers were incubated at different temperatures and for different periods of time. Then, the microparticles were tested on a single channel PRISM® instrument. The counts of four negative controls (NC) averages were used as a background. All samples used in study were listed in the table presented directly below. All samples were tested in duplicate. The Positive signal to Negative signal (P/N) ratio was used to evaluate the sensitivity.

| | | RIBA 3.0 | RIBA 3.0 | RIBA 3.0 |
|---|---|---|---|---|
| | | Test | Test | Test |
| | | Results | Results | Results |
| Panel/sample | Marker | c100 | 33c | c22 |
| Panel A | CORE | − | +/− | 3 |
| Panel B | NS3 | +/− | 2 | − |
| Panel F | CORE/NS3 | 1 | 2 | 3 |
| Sample 1 | CORE | − | +/− | 4 |

### EXAMPLE IV

### Testing Truncated HC43 (p9 MB18) After Incubation at 45° C. for 3 Days to Improve Anti-Core Antibody Detection

The p9 MB18 or p9 MB31, diluted with different microparticle diluents, was incubated at 45° C. for 3 days, then compared with the same microparticles stored at 2° C. to 8° C. for sensitivity of anti-core antibody detection. The results are presented in the table below.

The p9MB18-Coated Microparticles:

| | No Reductant | | 2 mM DTT | | 3 mM Cysteine | |
|---|---|---|---|---|---|---|
| Panel/ | 2° C. to | 45° C. | 2° C. to | 45° C. | 2° C. to | 45° C. |
| Samples | 8° C. | 3 days | 8° C. | 3 days | 8° C. | 3 days |
| NC (RLU*) | 43 | 33 | 32 | 33 | 32 | 33 |
| Panel A (P/N) | 5.42 | 12.5 | 5.73 | 6.83 | 10.7 | 10.5 |
| % Increase | | 131% | | 19% | | −2% |
| Sample 1 (P/N) | 4.33 | 18.8 | 2.72 | 3.94 | 11.2 | 12.3 |
| % Increase | | 334% | | 45% | | 10% |

*Relative Light Units

The results showed that when p9 MB18 microparticles were incubated at 45° C. for 3 days in the absence of reductant microparticle diluents, as shown above, the sensitivity of anti-core (Panel A and Sample 1) antibody detection was increased significantly.

The p9MB31-Coated Microparticles:

| | No Reductant | | 2 mM DTT | | 3 mM Cysteine | |
|---|---|---|---|---|---|---|
| Panel/ | 2° C. to | 45° C. | 2° C. to | 45° C. | 2° C. to | 45° C. |
| Samples | 8° C. | 3 days | 8° C. | 3 days | 8° C. | 3 days |
| NC (RLU) | 37 | 47 | 38 | 41 | 36 | 43 |
| Panel A (P/N) | 5.14 | 5.4 | 4 | 3.78 | 6.43 | 5.84 |
| % Increase | | 5% | | −6% | | −9% |

The p9 MB31 microparticles did not show the heat stress impact on anti-core antibody detection as shown by p9 MB18-coated microparticles.

### EXAMPLE V

### Truncated HC43 (p9 MB18): Effect of Temperature and Time on Anti-Core Antibody Detection

The p9 MB18-coated microparticles were suspended in different microparticle diluents as indicated in Example III and incubated at 37° C., 45° C., and 56° C. for up to 3 days. These microparticles were then tested with two anti-core positive samples (Panel A and Sample 1).

Microparticle Diluent Without Reductant:

| | 2° C. | 37° C. | | | 45° C. | | | 56° C. | | |
|---|---|---|---|---|---|---|---|---|---|---|
| Panel/ | to | 1 | 2 | 3 | 1 | 2 | 3 | 1 | 2 | 3 |
| Samples | 8° C. | day | day | day | day | day | day | day | day | day |
| NC (RLU) | 43 | 44 | 39 | 36 | 33 | 34 | 33 | 33 | 31 | 44 |
| Panel A (P/N) | 5.42 | 6.76 | 8.35 | 9.74 | 8.22 | 10.8 | 12.5 | 11.9 | 15.5 | 15.1 |
| % Increase | | 25% | 54% | 80% | 52% | 99% | 131% | 120% | 186% | 179% |
| Sample 1 (P/N) | 4.33 | 7.86 | 9.88 | 12.3 | 10.2 | 14.3 | 18.8 | 14.7 | 18.9 | 22.3 |
| % Increase | | 82% | 128% | 184% | 136% | 230% | 334% | 239% | 336% | 415% |

Microparticle Diluent with 2 mM DTT:

| | 2° C. | 37° C. | | | 45° C. | | | 56° C. | | |
|---|---|---|---|---|---|---|---|---|---|---|
| Panel/ | to | 1 | 2 | 3 | 1 | 2 | 3 | 1 | 2 | 3 |
| Samples | 8° C. | day | day | day | day | day | day | day | day | day |
| NC (RLU) | 32 | 30 | 30 | 31 | 33 | 33 | 33 | 33 | 35 | 32 |
| Panel A(P/N) | 5.73 | 8.15 | 6.3 | 7.32 | 6.11 | 6.18 | 6.83 | 6.72 | 6.1 | 7.27 |
| % Increase | | 42% | 10% | 28% | 7% | 8% | 19% | 17% | 6% | 27% |
| Sample 1(P/N) | 2.72 | 5.79 | 2.78 | 4.66 | 3.25 | 3.95 | 3.94 | 3.97 | 4.36 | 4.81 |
| % Increase | | 129% | 2% | 71% | 19% | 45% | 45% | 45% | 60% | 77% |

Microparticle Diluent With 3 mM Cysteine:

| | 2° C. | 37° C. | | | 45° C. | | | 56° C. | | |
|---|---|---|---|---|---|---|---|---|---|---|
| Panel/ | to | 1 | 2 | 3 | 1 | 2 | 3 | 1 | 2 | 3 |
| Samples | 8° C. | day | day | day | day | day | day | day | day | day |
| NC (RLU) | 32 | 30 | 30 | 31 | 33 | 33 | 33 | 33 | 35 | 32 |
| Panel A(P/N) | 10.7 | 9.61 | 10.8 | 13.2 | 9.41 | 11.9 | 10.5 | 9.63 | 11.2 | 13.1 |
| % Increase | | −10% | 0.1% | 23% | −12% | 11% | −2% | −10% | 5% | 22% |
| Sample 1(P/N) | 11.2 | 11.1 | 12 | 17.5 | 12.4 | 15.4 | 12.3 | 13.8 | 13.7 | 17.1 |
| % Increase | | −1% | 7% | 56% | 11% | 38% | 10% | 23% | 22% | 53% |

The results show that p9 MB18-coated microparticles in different microparticle diluents as shown above (with and without reductant), and heat stressed from 37° C. to 56° C. and up to 3 days, significantly improved the anti-core antibody detection.

### EXAMPLE VI

### Truncated HC43(p9 MB18 and p9 MB31): Effect of Different Microparticle Diluents and Temperature on Anti-NS₃ Antibody Detection

Both p9 MB18 and p9 MB31 coated microparticles were diluted with different microparticle diluent indicated in Example III, and incubated at 45° C. for 3 days. These microparticles were then compared with the same microparticles stored at 2° C. to 8° C., for sensitivity of anti-NS₃ antibody detection. Three NS₃ positive samples (Panel F, Panel B) were used in the test.

The p9MB18-Coated Microparticles:

| | No Reductant | | 2 mM DTT | | 3 mM Cysteine | |
|---|---|---|---|---|---|---|
| Panel/ | 2° C. to | 45° C. | 2° C. to | 45° C. | 2° C. to | 45° C. |
| Samples | 8° C. | 3 days | 8° C. | 3 days | 8° C. | 3 days |
| NC (RLU) | 40 | 45 | 38.5 | 37.5 | 37.5 | 39 |
| Panel F (P/N) | 6.33 | 4.86 | 5.79 | 7.19 | 6.86 | 4.56 |
| % Increase | | −23% | | 24% | | −34% |
| Panel B (P/N) | 11.23 | 7.61 | 11.65 | 13.33 | 11.73 | 8.45 |
| % Increase | | −32% | | 14% | | −28% |

The p9MB31-Coated Microparticles:

| | No Reductant | | 2 mM DTT | | 3 mM Cysteine | |
|---|---|---|---|---|---|---|
| Panel/ | 2° C. to | 45° C. | 2° C. to | 45° C. | 2° C. to | 45° C. |
| Samples | 8° C. | 3 days | 8° C. | 3 days | 8° C. | 3 days |
| NC (RLU) | 37 | 47 | 38 | 41 | 36 | 43 |
| Panel F (P/N) | 10.89 | 4.19 | 10.24 | 10.06 | 9.8 | 4.3 |
| % Increase | | −62% | | −1% | | −56% |
| Panel B (P/N) | 17.22 | 7.01 | 17.27 | 17.41 | 14.47 | 7.03 |
| % Increase | | −59% | | 1% | | −51% |

The results establish the following: 1) DTT reductant is required for stability of both p9 MB18- and p9 MB31-coated microparticles for the detection of anti-NS₃ antibody samples, and 2) DTT is superior to Cysteine as a reducing reagent.

Taking all three data sets together (as presented in the tables), one may summarize the results as follows:

1) Upon heat stress, p9 MB-18 showed overall higher sensitivity for Panel A and sample 1 compared to the non-heat stress condition.

2) Different reactivity was observed in connection with Panel A with respect to p9 MB-18 versus p9 MB-31 (i.e., different antigen constructs) when heat stressed at 45° C. for 3 days.

3) Upon heat stress, the antigens of panel B and F lost some sensitivity in the reductant free condition. However, both panels retained sensitivity in the presence of DTT. Moreover, upon use of the mild-reducing agent Cysteine, both panels lost some sensitivity on heat stress at 45 degrees Celsius for 3 days.

4) P9 MB-31-coated microparticles lost significant sensitivity for panels B and F, upon heat stress, in the absence of DTT and in the presence of Cysteine. Thus, in view of the above, the following conclusions can be reached: 1) p9 MB-18 showed better sensitivity for panel A (anti-core antibody sample) compared to p9 MB-31 on heat stress. 2) P9 MB-31-coated microparticles showed better sensitivity for panel B and F compared to the p9 MB-18-coated microparticles. 3) Heat stress at 45 degrees Celsius for 3 days resulted in better performance for p9 MB-18-coated microparticles when evaluated with Panel A and sample 1. DTT's presence resulted in better sensitivity for panel B and panel F when heat stressed at 45 degrees Celsius for 3 days with p9 MB-18-coated microparticles, and maintained the sensitivity for those two panels with p9 MB-31-coated microparticles. (Cysteine's presence did not improve stability for Panel B.) 4) Panel A and sample 1 showed better sensitivity with p9 MB-18-coated microparticles in the absence of DTT and in the presence of Cysteine at 2-8 degrees Celsius for 3 days, compared to placing the microparticles in the presence of DTT. Panel A showed better sensitivity with p9 MB-31-coated microparticles in the absence of DTT and in the presence of Cysteine at 2-8 degrees Celsius for 3 days, compared to placing the microparticles in the presence of DTT.

1. A method of simultaneously detecting at least one Hepatitis C Virus (HCV) antigen and at least one HCV antibody in a test sample comprising contacting said test sample with a mixture of:
a) at least one HCV antigen or fragment thereof coated on a solid phase, for a time and under conditions sufficient for the formation of antibody/antigen complexes, presence of said antibody/antigen complexes indicating presence of said at least one HCV antibody in said test sample; and
b) at least one HCV antibody or fragment thereof coated on said solid phase, for a time and under conditions sufficient for the formation of antigen/antibody complexes, presence of antigen/antibody complexes indicating presence of at least one HCV antigen in said test sample, wherein said method is carried out in the absence of reductant.

2. The method of claim 1 wherein said at least one HCV antigen coated on the solid phase is selected from the group consisting of core antigen, NS3, NS4, NS5, and fragments thereof.

3. The method of claim 2 wherein said at least one HCV antigen coated on the solid phase comprises amino acids 1192-1457 of NS3 and amino acids 1-31 and 51-100 of core protein, and said at least one antibody detected is selected from the group consisting of anti-NS3 antibody and anti-core antibody.

4. The method of claim 2 wherein said at least one HCV antigen coated on the solid phase comprises amino acids 1192-1457 of NS3 and amino acids 1-31, 34-46 and 49-100 of core protein, and said at least one antibody detected is selected from the group consisting of anti-NS3 antibody and anti-core antibody.

5. The method of claim 1 wherein said solid phase is a microparticle.

6. The method of claim 1 wherein said method further comprises the step of applying heat subsequent to said contacting step.

7. The method of claim 6 wherein said at least one antigen coated on said solid phase comprises amino acids 1192-1457 of NS3 and amino acids 1-31 and 51-100 of core protein and said at least one antibody detected is anti-core antibody.

8. A method for simultaneously detecting the presence of at least one HCV antigen and at least one HCV antibody in a test sample comprising the steps of:
a) contacting said test sample with: 1) at least one HCV antigen or fragment thereof coated on a solid phase, for a time and under conditions sufficient for the formation of antibody/antigen complexes and 2) at least one HCV antibody or portion thereof coated on said solid phase, wherein said first antibody is anti-HCV antibody, for a time and under conditions sufficient for the formation of antigen/antibody complexes;
b) adding a conjugate to the resulting antibody/antigen complexes for a time and under conditions sufficient to allow said conjugate to bind to the bound antibody in (a)(1), wherein said conjugate comprises an antibody attached to a chemiluminescent compound capable of generating a detectable signal and simultaneously adding a second conjugate to the resulting antigen/antibody complexes for a time and under conditions sufficient to allow said conjugate to bind to the bound antigen in (a)(2), wherein said conjugate comprises an antibody attached to said chemiluminescent compound capable of generating a detectable signal; and
c) detecting said generated signal, presence of said signal indicating presence of at least one HCV antigen, at least one HCV antibody, or both, in said test sample,
wherein said method is carried out in the absence of a reductant.

9. The method of claim 8 wherein said at least one HCV antigen coated on the solid phase is selected from the group consisting of core antigen, NS3, NS4, NS5, and fragments thereof.

10. The method of claim 9 wherein said at least one antigen coated on the solid phase comprises amino acids 1192-1457 of NS3 and amino acids 1-31 and 51-100 of core protein and said at least one antibody detected is selected from the group consisting of anti-NS3 antibody and anti-core antibody.

11. The method of claim 9 wherein said at least one antigen coated on the solid phase comprises amino acids 1192-1457 of NS3 and amino acids 1-30, 34-46 and 49-100 amino acids of core protein and said at least one antibody detected is selected from the group consisting of anti-NS3 antibody and anti-core antibody.

12. The method of claim 8 wherein said method further comprises applying heat subsequent to step a).

13. The method of claim 12 wherein said at least one antigen coated on the solid phase comprises amino acids 1192-1457 of NS3 and amino acids 1-31 and 51-100 of core protein and said at least one antibody detected is anti-core antibody.

14. A method of simultaneously detecting at least one Hepatitis C Virus (HCV) antigen and at least one HCV antibody in a test sample comprising contacting said test sample with a mixture of:
a) at least one HCV antigen or fragment thereof coated on a solid phase, for a time and under conditions sufficient for the formation of antibody/antigen complexes, presence of said antibody/antigen complexes indicating presence of said at least one HCV antibody in said test sample; and
b) at least one HCV antibody or fragment thereof coated on said solid phase, for a time and under conditions sufficient for the formation of antigen/antibody complexes, presence of antigen/antibody complexes indicating presence of at least one HCV antigen in said test sample, wherein said method is carried out in the presence of a reductant selected from the group consisting of Dithiothreitol (DTT) and Cysteine.

15. The method of claim 14 wherein said at least one HCV antigen coated on the solid phase is selected from the group consisting of core antigen, NS3, NS4, NS5, and fragments thereof.

16. The method of claim 15 wherein said at least one antigen coated on said solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

17. The method of claim 15 wherein said at least one antigen coated on said solid phase comprising amino acids 1192-1457 of NS3 and amino acids 1-30, 34-46 and 49-100 of core protein, and said at least one antibody detected is selected from the group consisting of anti-NS3 antibody and anti-core antibody, and said reductant is DTT.

18. The method of claim 14 further comprising the step of applying heat subsequent to said contacting step.

19. The method of claim 18 wherein said at least one antigen coated on said solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

20. A method for simultaneously detecting the presence of at least one HCV antigen and at least one HCV antibody in a test sample comprising the steps of:
a) contacting said test sample with: 1) at least one HCV antigen or fragment thereof coated on a solid phase, for a time and under conditions sufficient for the formation of antibody/antigen complexes and 2) at least one first antibody or fragment thereof coated on said solid phase, wherein said first antibody is anti-HCV antibody, for a time and under conditions sufficient for the formation of antigen/antibody complexes;
b) adding a first conjugate to the resulting antibody/antigen complexes for a time and under conditions sufficient to allow said first conjugate to bind to the bound antibody in (a)(1), wherein said first conjugate comprises an antibody attached to a chemiluminescent compound capable of generating a detectable signal and simultaneously adding a second conjugate to the resulting antigen/antibody complexes for a time and under conditions sufficient to allow said second conjugate to bind to the bound antigen in (a)(2), wherein said second conjugate comprises an antibody attached to said chemiluminescent compound capable of generating a detectable signal; and
c) detecting said generated signal, presence of said signal indicating presence of said at least one HCV antigen, said at least one HCV antibody, or both in said test sample,
wherein said method is carried out in the presence of a diluent comprising a reductant selected from the group consisting of DTT and Cysteine.

21. The method of claim 20 wherein said at least one HCV antigen coated on the solid phase is selected from the group consisting of core antigen, NS3, NS4, NS5, and fragments thereof.

22. The method of claim 21 wherein said at least one antigen coated on the solid phase comprises amino acids 1192-1457 of NS3 and amino acids 1-31 and 51-100 of core protein, said at least one antibody detected is selected from the group consisting of anti-NS3 antibody and anti-core antibody, and said reductant is D

23. The method of claim 21 wherein said at least one antigen coated on the solid phase comprises amino acids 1192-1457 of NS3 and amino acids 1-30, 34-46 and 49-100 of core protein, said at least one antibody is selected from the group consisting of anti-NS3 antibody and anti-core antibody, and said reductant is DTT.

24. The method of claim 20 further comprising the step of applying heat subsequent to step (a).

25. The method of claim 24 wherein said at least one antigen coated on the solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

26. A method of detecting at least one HCV antibody in a test sample comprising contacting said test sample with at least one HCV antigen or fragment thereof coated on a solid phase, for a time and under conditions sufficient for the formation of antibody/antigen complexes, presence of said antibody/antigen complexes indicating presence of said at least one HCV antibody in said test sample, wherein said method is carried out in the absence of reductant.

27. The method of claim 26 wherein said at least one HCV antigen coated on the solid phase is selected from the group consisting of core antigen, NS3, NS4, NS5, and fragments thereof.

28. The method of claim 27 wherein said at least one HCV antigen coated on the solid phase comprises amino acids 1192-1457 of NS3 and amino acids 1-31 and 51-100 of core protein, and said at least one antibody detected is selected from the group consisting of anti-NS3 antibody and anti-core antibody.

29. The method of claim 27 wherein said at least one HCV antigen coated on the solid phase comprises amino acids 1192-1457 of NS3 and amino acids 1-31, 34-46 and 49-100 of core protein, and said at least one antibody detected is selected from the group consisting of anti-NS3 antibody and anti-core antibody.

30. The method of claim 26 wherein said solid phase is a microparticle.

31. The method of claim 26 wherein said method further comprises the step of applying heat subsequent to said contacting step.

32. The method of claim 26 wherein said at least one antigen coated on said solid phase comprises amino acids 1192-1457 of NS3 and amino acids 1-31 and 51-100 of core protein and said at least one antibody detected is anti-core antibody.

33. A method of detecting at least one HCV antibody in a test sample comprising the steps of:
a) contacting said test sample with at least one HCV antigen or fragment thereof coated on a solid phase, for a time and under conditions sufficient for the formation of antibody/antigen complexes;
b) adding a conjugate to the resulting antibody/antigen complexes for a time and under conditions sufficient to allow said conjugate to bind to the bound antibody in (a), wherein said conjugate comprises an antibody attached to a chemiluminescent compound capable of generating a detectable signal; and
c) detecting said generated signal, presence of said signal indicating presence of said at least one HCV antibody in said test sample,
wherein said method is carried out in the absence of a reductant.

34. The method of claim 33 wherein said at least one HCV antigen coated on the solid phase is selected from the group consisting of core antigen, NS3, NS4, NS5, and fragments thereof.

35. The method of claim 34 wherein said at least one antigen coated on the solid phase comprises amino acids 1192-1457 of NS3 and amino acids 1-31 and 51-100 of core protein and said at least one antibody detected is selected from the group consisting of anti-NS3 antibody and anti-core antibody.

36. The method of claim 34 wherein said at least one antigen coated on the solid phase comprises amino acids 1192-1457 of NS3 and amino acids 1-30, 34-46 and 49-100 amino acids of core protein and said at least one antibody detected is selected from the group consisting of anti-NS3 antibody and anti-core antibody.

37. The method of claim 33 wherein said method further comprises applying heat subsequent to step (a).

38. The method of claim 37 wherein said at least one antigen coated on the solid phase comprises amino acids 1192-1457 of NS3 and amino acids 1-31 and 51-100 of core protein and said at least one antibody detected is anti-core antibody.

39. A method of detecting at least one HCV antibody in a test sample comprising contacting said test sample with at least one HCV antigen or fragment thereof coated on a solid phase, for a time and under conditions sufficient for the formation of antibody/antigen complexes, presence of said antibody/antigen complexes indicating presence of said at least one HCV antibody in said test sample, wherein said method is carried out in the presence of a reductant selected from the group consisting of DTT and Cysteine.

40. The method of claim 39 wherein said at least one HCV antigen coated on the solid phase is selected from the group consisting of core antigen, NS3, NS4 and NS5, and fragments thereof.

41. The method of claim 40 wherein said at least one antigen coated on said solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

42. The method of claim 40 wherein said at least one antigen coated on said solid phase comprising amino acids 1192-1457 of NS3 and amino acids 1-30, 34-46 and 49-100 of core protein, and said at least one antibody detected is selected from the group consisting of anti-NS3 antibody and anti-core antibody, and said reductant is DTT.

43. The method of claim 39 further comprising the step of applying heat subsequent to said contacting step.

44. The method of claim 43 wherein said at least one antigen coated on said solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

45. A method for detecting the presence of at least one HCV antibody in a test sample comprising the steps of:
a) contacting said test sample with at least one HCV antigen or fragment thereof coated on a solid phase, for a time and under conditions sufficient for the formation of antibody/antigen complexes;
b) adding a conjugate to the resulting antibody/antigen complexes for a time and under conditions sufficient to allow said conjugate to bind to the bound antibody in (a), wherein said conjugate comprises an antibody attached to a chemiluminescent compound capable of generating a detectable signal; and
c) detecting said generated signal, presence of said signal indicating presence of at least one HCV antibody in said test sample, wherein said method is carried out in the presence of a diluent comprising a reductant selected from the group consisting of DTT and Cysteine.

46. The method of claim 45 wherein said at least one HCV antigen coated on the solid phase is selected from the group consisting of core antigen, NS3, NS4, NS5, and fragments thereof.

47. The method of claim 46 wherein said at least one antigen coated on the solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

48. The method of claim 46 wherein said at least one antigen coated on the solid phase comprises amino acids 1192-1457 of NS3 and amino acids 1-30, 34-46 and 49-100 of core protein, said at least one antibody is selected from the group consisting of anti-NS3 antibody and anti-core antibody, and said reductant is DTT.

49. The method of claim 45 further comprising the step of applying heat subsequent to step (a).

50. The method of claim 49 wherein said at least one antigen coated on the solid phase comprises amino acids 1192-1457 of NS3 and amino acids 1-31 and 51-100 of core protein, said at least one antibody detected is selected from the group consisting of anti-NS3 antibody and anti-core antibody, and said reductant is DTT.

51. A kit comprising:
a) a container containing at least one HCV antigen coated on a solid phase, wherein said container contains a diluent lacking a reductant; and
b) a container containing at least one HCV antibody coated on a solid phase.

52. A kit comprising:
a container containing: 1) at least one HCV antigen coated on a solid phase and 2) at least one HCV antibody, coated on said solid phase, wherein said container contains a diluent lacking a reductant.
